(19) United States
(12) Patent Application Publication
(10) Pub. No.: US 2026/0076796 A1
(43) Pub. Date: Mar. 19, 2026

(54) REPLACEMENT HEART VALVE IMPLANT WITH LEAFLET PINNING EXPANDABLE FRAMEWORK

(71) Applicant: BOSTON SCIENTIFIC SCIMED, INC., MAPLE GROVE, MN (US)

(72) Inventors: Shane Conway, Longford (IE); Scott Cook, Athenry (IE); David Ryan, Claremorris (IE)

(73) Assignee: BOSTON SCIENTIFIC SCIMED, INC., MAPLE GROVE, MN (US)

(21) Appl. No.: 19/331,530

(22) Filed: Sep. 17, 2025

Related U.S. Application Data
(60) Provisional application No. 63/696,037, filed on Sep. 18, 2024.

(51) Int. Cl. A61F 2/24

(52) U.S. Cl. CPC A61F 2/2418; A61F 2230/0006; A61F 2230/0017; A61F 2230/0054; A61F 2230/0058

(57) ABSTRACT

An expandable framework for use in a replacement heart valve implant includes a plurality of struts defining a lattice structure with an inflow end, an outflow end, and a plurality of stabilization arches extending downstream from the outflow end. The lattice structure includes a first circumferential row of cells defining a plurality of lower crowns at the inflow end and a second circumferential row of cells defining a plurality of upper crowns at the outflow end, where the cells of the first and second circumferential rows each have a lower point and an upper point. The lower point of at least some of the cells in the second circumferential row is a free point devoid of attachment to any other strut and extends radially outward beyond a radially outer surface of any strut immediately below in an axial direction.

### CROSS-REFERENCE TO RELATED APPLICATIONS

This application claims the benefit of priority of U.S. Provisional Application No. 63/696,037 filed Sep. 18, 2024, the entire disclosure of which is hereby incorporated by reference.

### TECHNICAL FIELD

The present disclosure pertains to medical devices, systems, and methods for manufacturing and/or using medical devices and/or systems. More particularly, the present disclosure pertains to a replacement heart valve implant and/or an expandable framework for use in a replacement heart valve implant.

### BACKGROUND

A wide variety of intracorporal medical devices have been developed for medical use, for example, intravascular use. Some of these devices include guidewires, catheters, medical device systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example expandable framework for use in a replacement heart valve implant includes a plurality of struts defining a lattice structure around a central longitudinal axis, the lattice structure defining an inflow end and an opposite outflow end, the plurality of struts defining a plurality of stabilization arches extending downstream from the outflow end of the lattice structure, wherein the plurality of struts defines a plurality of lower crowns defining the inflow end and a plurality of upper crowns defining the outflow end, wherein the lattice structure includes a first circumferential row of cells defining the lower crowns and the inflow end and a second circumferential row of cells defining the upper crowns and the outflow end, wherein the cells of the first and second circumferential rows each have a lower point and an upper point, and wherein the lower point of at least some of the cells in the second circumferential row is a free point devoid of attachment to any other strut and extends radially outward beyond a radially outer surface of any strut immediately below in an axial direction.

Alternatively or additionally to the embodiment above, some of the lower points of the cells in the first circumferential row define the lower crowns and some of the upper points of the cells in the second circumferential row define the upper crowns, wherein the upper points defining upper crowns are bent radially outward.

Alternatively or additionally to any of the embodiments above, three or more free points are spaced apart around the lattice structure.

Alternatively or additionally to any of the embodiments above, an outer surface of each free point extends radially outward 2.0 mm (0.079 inches) to 6.0 mm (0.0236 inches) beyond the radially outer surface of any strut below in the axial direction.

Alternatively or additionally to any of the embodiments above, an axial gap of 0.7 mm (0.028 inches) to 2 mm (0.079 inches) is defined between each free point and the strut immediately below in the axial direction.

Alternatively or additionally to any of the embodiments above, each free point extends at an angle of 20 degrees to 60 degrees from the central longitudinal axis of the expandable framework.

Alternatively or additionally to any of the embodiments above, the cells in the first and second circumferential rows are diamond shaped with upper left and right struts meeting at the upper point of each cell and lower left and right struts meeting at the lower point of each cell, wherein some cells in the first circumferential row have a right side connection point joining the upper and lower right struts and a left side connection point joining the upper and lower left struts, where the right side connection point is also joined with the left side connection point of a circumferentially adjacent cell to form an X shaped join, wherein some circumferentially adjacent cells in the first circumferential row are devoid of the X shaped join wherein the upper right strut of a first cell is joined only with the upper left strut of an adjacent second cell, and the lower right strut of the first cell is joined only with the lower left strut of the second cell such that the joined upper right and left struts of the first and second cells defines the free point of one of the cells in the second circumferential row.

Alternatively or additionally to any of the embodiments above, some of the lower points of the cells in the first circumferential row define the lower crowns and wherein some of the upper points of the cells in the second circumferential row define the upper crowns, wherein the upper points defining upper crowns are bent radially outward.

Alternatively or additionally to any of the embodiments above, three or more free points are spaced apart around the lattice structure.

Alternatively or additionally to any of the embodiments above, an outer surface of each free point extends radially outward 2.0 mm (0.079 inches) to 6.0 mm (0.236 inches) beyond the radially outer surface of any strut immediately below in the axial direction.

Alternatively or additionally to any of the embodiments above, an axial gap of 0.7 mm (0.028 inches) to 2 mm (0.079 inches) is defined between each free point and the strut immediately below in the axial direction.

Alternatively or additionally to any of the embodiments above, each free point extends at an angle of 20 degrees to 60 degrees from the central longitudinal axis of the expandable framework.

Alternatively or additionally to any of the embodiments above, the expandable framework further comprises a third circumferential row of cells between and circumferentially offset from the first and second circumferential rows of cells, wherein the cells in the first, second and third circumferential rows are diamond shaped with a majority of cells having upper left and right struts meeting at the upper point of the cell and left and lower right struts meeting at the lower point of the cell, wherein the upper left and right struts of each cell in the first circumferential row are the lower right and left struts, respectively, of adjacent cells in the third circumferential row, wherein at least a first cell in the first circumferential row is devoid of upper left and right struts such that the first cell defines a V-shaped cell with first and second adjacent cells in the third circumferential row, and the free point is defined by the lower point of a first cell in the second circumferential row axially aligned with the first cell in the first circumferential row.

Alternatively or additionally to any of the embodiments above, at least three free points are spaced apart around the lattice structure.

Alternatively or additionally to any of the embodiments above, an outer surface of each free point extends radially outward 2.0 mm (0.079 inches) to 6.0 mm (0.236 inches) beyond the radially outer surface of any strut immediately below in the axial direction.

Alternatively or additionally to any of the embodiments above, each free point extends at an angle of 20 degrees to 60 degrees from the central longitudinal axis of the expandable framework.

An example replacement heart valve comprises an expandable framework including a plurality of struts defining at least first and second circumferential rows of diamond shaped cells around a central longitudinal axis defining an inflow end and an opposite outflow end, the plurality of struts defining a plurality of stabilization arches extending downstream from the outflow end, wherein the first circumferential row of cells defines a plurality of lower crowns at the inflow end, and the second circumferential row of cells defines a plurality of upper crowns at the outflow end, wherein the cells of the first and second circumferential rows each have a lower point and an upper point, and wherein the lower point of at least some of the cells in the second circumferential row is a free point devoid of attachment to any other strut and extends radially outward beyond a radially outer surface of any strut immediately below in an axial direction.

Alternatively or additionally to the embodiment above, the diamond shaped cells in the first and second circumferential rows have upper left and right struts meeting at the upper point of the cell and lower left and right struts meeting at the lower point of the cell, wherein some cells in the first circumferential row have a right side connection point joining the upper and lower right struts and a left side connection point joining the upper and lower left struts, where the right side connection point is also joined with the left side connection point of a circumferentially adjacent cell to form an X shaped join, wherein some circumferentially adjacent cells in the first circumferential row are devoid of the X shaped join wherein the upper right strut of a first cell is joined only with the upper left strut of an adjacent second cell, and the lower right strut of the first cell is joined only with the lower left strut of the second cell such that the joined upper right and left struts of the first and second cells defines the free point of one of the cells in the second circumferential row.

Alternatively or additionally to any of the embodiments above, the replacement heart valve further comprises a third circumferential row of cells between and circumferentially offset from the first and second circumferential rows of cells, wherein the cells in the first, second and third circumferential rows are diamond shaped with a majority of cells having upper left and right struts meeting at the upper point of the cell and lower left and right struts meeting at the lower point of the cell, wherein the upper left and right struts of each cell in the first circumferential row are the lower right and left struts, respectively, of adjacent cells in the third circumferential row, wherein at least a first cell in the first circumferential row is devoid of upper left and right struts such that the first cell defines a V-shaped cell with first and second adjacent cells in the third circumferential row, and the free point is defined by the lower point of a first cell in the second circumferential row axially aligned with the first cell in the first circumferential row.

An example method of implanting a replacement heart valve within a patient's native heart valve, comprising advancing a replacement heart valve through a patient's vasculature and upstream into the patient's native heart valve, the replacement heart valve including an expandable framework and replacement valve secured thereto, the expandable framework including a plurality of struts defining a lattice structure around a central longitudinal axis, the lattice structure defining an inflow end and an opposite outflow end, the plurality of struts defining a plurality of stabilization arches extending downstream from the outflow end of the lattice structure, wherein the plurality of struts defines a plurality of lower crowns defining the inflow end and a plurality of upper crowns defining the outflow end, wherein the lattice structure includes a first circumferential row of cells defining the lower crowns and the inflow end and a second circumferential row of cells defining the upper crowns and the outflow end, wherein the cells of the first and second circumferential rows each have a lower point and an upper point, wherein the lower point of at least some of the cells in the second circumferential row is a free point devoid of attachment to any other strut and extends radially outward beyond a radially outer surface of all struts immediately below in an axial direction, and positioning the expandable framework to pin at least one leaflet of the native heart valve between the free point and the struts below the free point.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates selected aspects of a native heart valve;

FIG. 2 illustrates a prior art replacement heart valve;

FIG. 3 illustrates selected aspects of an example expandable framework for use in a replacement heart valve implant;

FIG. 4 is a partial cross-sectional view of FIG. 3;

FIG. 5 is a top down view of the expandable framework of FIG. 3;

FIG. 6 is a partial cross-sectional view of another expandable framework for use in a replacement heart valve implant;

FIG. 7 is a close-up view of the free point of the expandable framework of FIG. 3; and

FIG. 8 is a partial cross-sectional view of the expandable framework of FIG. 6 positioned within the native heart valve.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate example embodiments of the disclosure but not limit the disclosure. Those skilled in the art will recognize that the various elements described and/or shown may be arranged in various combinations and configurations without departing from the scope of the disclosure. However, in the interest of clarity and ease of understanding, every feature and/or element may not be shown in each drawing.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage.

Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently - such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Diseases and/or medical conditions that impact the cardiovascular system are prevalent throughout the world. Some mammalian hearts (e.g., human, etc.) include four heart valves: a tricuspid valve, a pulmonary valve, an aortic valve, and a mitral valve. Some relatively common medical conditions may include or be the result of inefficiency, ineffectiveness, or complete failure of one or more of the valves within the heart. For example, failure of the aortic valve or the mitral valve can have a serious effect on a human and could lead to a serious health condition and/or death if not dealt with properly. Treatment of defective heart valves poses other challenges in that the treatment often requires the repair or outright replacement of the defective heart valve. Such therapies may be highly invasive to the patient. Disclosed herein is an apparatus, system, and/or method that may be used in a portion of the cardiovascular system in order to diagnose, treat, and/or repair the system. In some embodiments, the apparatus, system, and/or method disclosed herein may be used during a procedure to diagnose, treat, and/or repair a defective heart valve (e.g., the aortic valve, the mitral valve, etc.). In addition, a replacement heart valve implant may be delivered percutaneously and thus may be much less invasive to the patient. The apparatus, system, and/or method disclosed herein may also provide other desirable features and/or benefits as described below.

It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to “the leaflet”, “the strut”, or other features may be equally referred to all instances and quantities beyond one of said feature unless clearly stated to the contrary. As such, it will be understood that the following discussion may apply equally to any and/or all of the components for which there are more than one within the replacement heart valve implant and/or the apparatus unless explicitly stated to the contrary.

Additionally, it should be noted that in any given figure, some features may not be shown, or may be shown schematically, for clarity and/or simplicity. Additional details regarding some components and/or method steps may be illustrated in other figures in greater detail. The systems, devices, and/or methods disclosed herein may provide a number of desirable features and benefits as described in more detail below. For the purpose of this disclosure, the discussion below is directed toward the treatment of a native aortic valve and will be so described in the interest of brevity. This, however, is not intended to be limiting as the skilled person will recognize that the following discussion may also apply to a mitral valve or another heart valve with no or minimal changes to the structure and/or scope of the disclosure. Similarly, the medical devices disclosed herein may have applications and uses in other portions of a patient's anatomy, such as but not limited to, arteries, veins, and/or other body lumens.

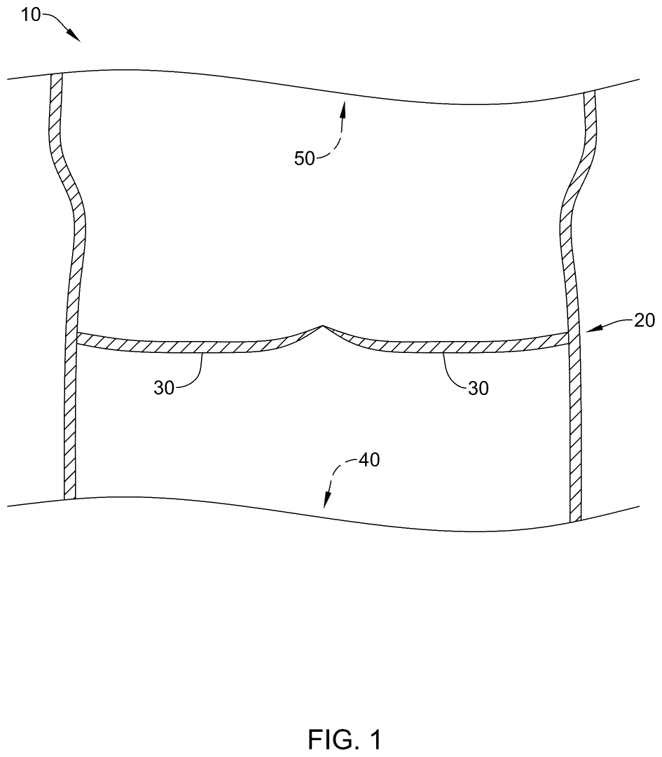

FIG. 1 illustrates a native heart valve 10 (e.g., a native aortic valve, etc.) in a closed position. The native heart valve 10 may include an annulus 20 defined at least partially by one or more walls of the native heart, and a plurality of leaflets 30 extending radially inward from the annulus 20. During systole, the plurality of leaflets 30 may shift to an open position (move upwards in the figure) to permit blood to flow through the native heart valve 10 (e.g., from the left ventricle 40, through the native heart valve 10, and downstream into the aortic arch 50). During diastole, the plurality of leaflets 30 may shift to a closed position to prevent blood from flowing through the native heart valve 10. In some cases, a diseased and/or defective native heart valve 10 may have and/or include calcification in, on, and/or around the annulus 20.

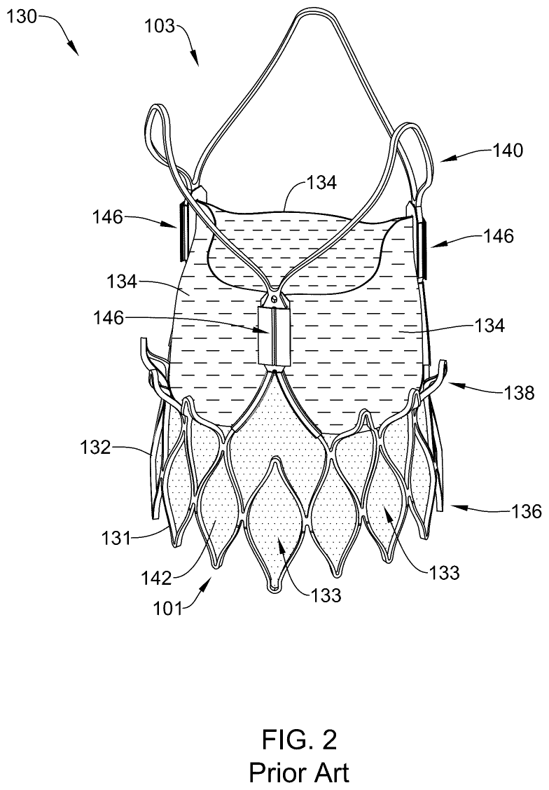

FIG. 2 illustrates selected aspects of a prior art replacement heart valve implant 130. The replacement heart valve implant 130 includes an expandable framework 132 formed by a plurality of struts 131 defining a central lumen. The side of the expandable framework 132 and other components facing the central lumen are referred to as the luminal surface. The opposite or outer side of the expandable framework 132 and other components (e.g., facing away from the central lumen) are referred to as the abluminal surface. The plurality of struts 131 defines a plurality of cells 133 (e.g., openings) between adjacent struts and extending through the expandable framework 132 from the luminal side to the abluminal side. The plurality of struts 131 defines a plurality of lower crowns 136 proximate the inflow end 101 of the lattice structure, a plurality of upper crowns 138 proximate the outflow end 103 of the lattice structure, and a plurality of stabilization arches 140 extending downstream from the outflow end of the lattice structure. The replacement heart valve implant 130 includes a plurality of valve leaflets 134 disposed within the central lumen. The plurality of valve leaflets 134 are coupled, secured, and/or fixedly attached to the expandable framework 132. Each of the plurality of valve leaflets 134 includes two connection portions that contact the expandable framework 132 at a plurality of commissures 146 disposed adjacent the plurality of stabilization arches 140. The plurality of valve leaflets 134 may be formed integrally with other structures such as an inner skirt 142 and/or an outer skirt (not shown).

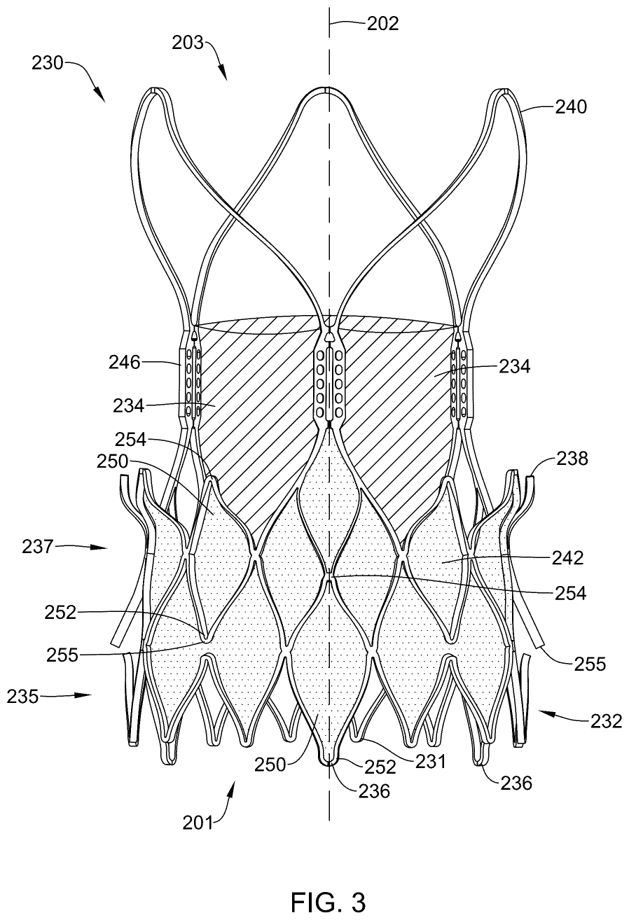

In cases where a patient exhibits aortic insufficiency (AI) and/or aortic regurgitation (AR), additional framework structures may be desired to increase the force and anchoring capability of the replacement heart valve against the patient's anatomy. FIG. 3 illustrates an example of such an expandable framework 232 in a replacement heart valve implant 230. The expandable framework 232 is configured to pin a leaflet of the native heart valve against the outer surface of the expandable framework. The replacement heart valve implant 230 may include a plurality of valve leaflets 234 coupled, secured, and/or fixedly attached to the expandable framework 232. Each of the plurality of valve leaflets 234 may include a root edge coupled to the expandable framework 232 and a free edge (e.g., a coaptation edge) movable relative to the root edge to coapt with the coaptation edges of the other leaflets along a coaptation region. In some embodiments, the plurality of valve leaflets 234 can be integrally formed with each other, such that the plurality of valve leaflets 234 is formed as a single unitary and/or monolithic unit. In some embodiments, a “root edge” can be a formed edge, such as when the plurality of valve leaflets 234 is formed in place on the expandable framework 232. In some embodiments, the plurality of valve leaflets 234 may be formed integrally with other structures such as an inner skirt 242 and/or an outer skirt (not shown), base structures, liners, or the like and in those circumstances the “root edge” is not a cut or otherwise divided edge, but rather is the location opposite the free edge where each of the plurality of valve leaflets 234 contacts those other structures.

Each of the plurality of valve leaflets 234 may further include two connection portions. One connection portion can be disposed on either end of the free edge of its respective leaflet such that the connection portions are contacting or adjacent to the expandable framework 232 at a plurality of commissures 246 disposed adjacent a plurality of stabilization arches 240. In some embodiments, the plurality of valve leaflets 234 may be secured and/or fixedly attached to the expandable framework 232 at the plurality of commissures 246 disposed adjacent the plurality of stabilization arches 240. The free edges of the plurality of valve leaflets 234 may extend between the plurality of commissures 246.

In some embodiments, the plurality of commissures 246 may be disposed at a base of the plurality of stabilization arches 240. In some embodiments, each of the plurality of commissures 246 may join circumferentially adjacent stabilization arches of the plurality of stabilization arches 240 together. In some embodiments, the plurality of commissures 246 may be disposed longitudinally between the plurality of stabilization arches 240 and a plurality of upper crowns 238. In some embodiments, the plurality of commissures 246 may be disposed distal of the plurality of stabilization arches 240 and proximal of the plurality of upper crowns 238. In at least some embodiments, between circumferentially adjacent commissures of the plurality of commissures 246, the replacement heart valve implant 230 may be devoid of the expandable framework 232 at a longitudinal position radially outward of the free edges of the plurality of valve leaflets 234. As such, the free edges of the plurality of valve leaflets 234 may be free from direct contact with the expandable framework 232 as the plurality of valve leaflets 234 opens and/or closes.

The replacement heart valve implant 230 is configured to allow one-way flow through the replacement heart valve implant from an inflow end 201 to an outflow end 203. The expandable framework 232 may be configured to shift from a collapsed configuration to an expanded configuration. In some embodiments, the expandable framework 232 may be self-expanding. In some embodiments, the expandable framework 232 may be self-biased toward the expanded configuration. In some embodiments, the expandable framework 232 may be mechanically expandable. In some embodiments, the expandable framework 232 may be balloon expandable. Other configurations are also contemplated.

The expandable framework 232 may include a plurality of struts 231 that define a lattice structure disposed and/or extending around a central longitudinal axis 202. The lattice structure may define the inflow end 201 and the opposite outflow end 203. The plurality of struts 231 may define the plurality of stabilization arches 240 extending downstream from the outflow end of the lattice structure. The plurality of struts 231 may define the plurality of lower crowns 236 defining the inflow end 201 and the plurality of upper crowns 238 defining the outflow end 203 of the lattice structure. The lattice structure may include a first circumferential row 235 of cells 250 defining the lower crowns 236 and the inflow end 201 and a second circumferential row 237 of cells 250 defining the upper crowns 238 and the outflow end of the lattice structure. The cells 250 of the first and second circumferential rows 235, 237 each have a lower point 252 and an upper point 254. The lower points 252 of every cell 250 in the first circumferential row 235 are free points devoid of attachment to any other strut 231. The upper points 254 of some cells 250 in the second circumferential row 237 join with additional struts 231 forming the commissures 246 and the stabilization arches 240. The lower point of at least some of the cells 250 in the second circumferential row 237 is a free point 255 devoid of attachment to any other strut and extends radially outward beyond a radially outer surface of any strut 231 immediately below in an axial direction. The free point 255 is configured to trap or pin a leaflet of the native heart valve against the lower struts of the expandable framework. The free points 255 may apply a greater force to the anatomy, and this increased force may increase the total radial force applied by the expandable framework 232 on the anatomy to reduce or prevent movement of the expandable framework and the replacement valve including the framework.

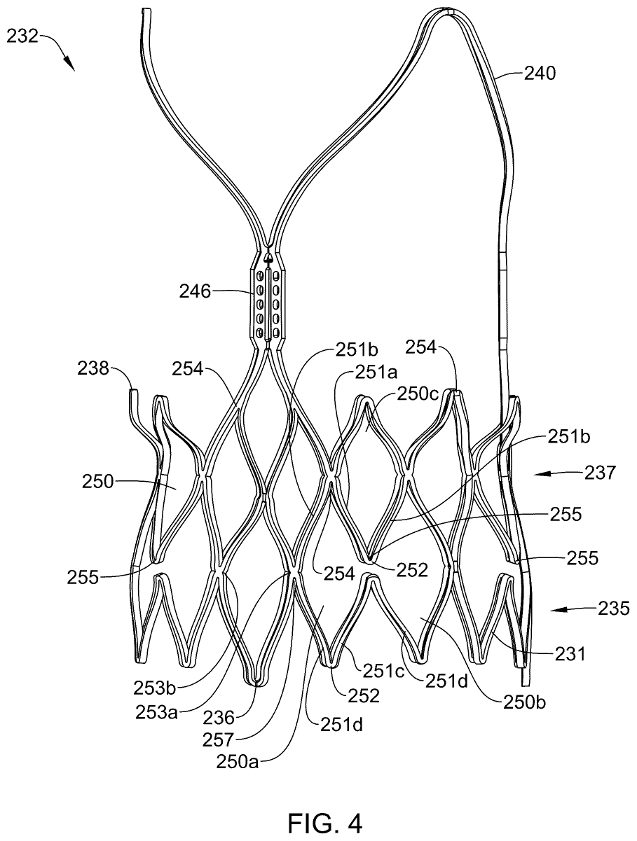

In some embodiments, some of the lower points 252 of the cells 250 in the first circumferential row 235 define the lower crowns 236 and some of the upper points 254 of the cells in the second circumferential row 237 define the upper crowns 238, and the upper points 254 defining upper crowns 238 are bent radially outward. The expandable framework 232 may include at least three free points 255 spaced apart around the lattice structure. The free points 255 may be defined in circumferentially alternating cells. In one embodiment, two free points 255 are defined under each stabilization arch 240, as shown in FIGS. 3 and 4. This embodiment has three stabilization arches and six total free points 255. In other embodiments, a single free point may be defined under each stabilization arch, or three free points may be defined under each stabilization arch. In still further embodiments, the free points 255 may be defined in positions other than under the stabilization arches, such as below the commissures 246.

The embodiment shown in FIGS. 3-5 illustrate a first expandable framework 232 defining a first configuration of free points 255. The partial cross-sectional view in FIG. 4 shows more details of the structure in FIG. 3. The cells 250 in the first and second circumferential rows 235, 237 of the expandable framework 232 may be diamond shaped with an upper right strut 251a and an upper left strut 251b meeting to form the upper point 254 of the cell and a lower right strut 251c and a lower left strut 251d meeting to form the lower point 252 of the cell. In the embodiment shown in FIGS. 3 and 4, the expandable framework 232 is formed by only first and second circumferential rows 235, 237 of cells 250. Thus, the lower right and left struts of a cell in the second circumferential row 237 are the same struts as the upper left and right struts, respectively, of two adjacent cells in the first circumferential row 235. Some cells in the first circumferential row 235 have a right side connection point 253a joining the upper and lower right struts 251a, 251c and a left side connection point 253b joining the upper and lower left struts 251b, 251d. The right side connection point 253a is also joined with the left side connection point 253b of a circumferentially adjacent cell to form an X shaped join 257. Some circumferentially adjacent cells in the first circumferential row 235 are devoid of the X shaped join such that the upper right strut 251a of a first cell 250a is joined only with the upper left strut 251b of an adjacent second cell 250b and the lower right strut 251c of the first cell 250a is joined only with the lower left strut 251d of the second cell 250b such that the joined upper right and left struts 251a, 251b of the first and second cells 250a, 250b defines the free point 255 of a cell 250c in the second circumferential row 237. In some embodiments, one or more additional circumferential rows of cells may be provided and/or disposed longitudinally between the first and second circumferential rows 235, 237.

In the embodiment shown in FIG. 4, the lower point 252 of some cells 250 in the second row is a free point 255, while the lower point 252 of other cells in the second row is joined with side connection points 253a, 253b of two adjacent cells 250 in the first circumferential row 235. The upper point 254 of some cells 250 in the second circumferential row 237 are free and form the upper crowns 238 and other upper points 254 join with struts forming the commissures 246 and stabilization arches 240.

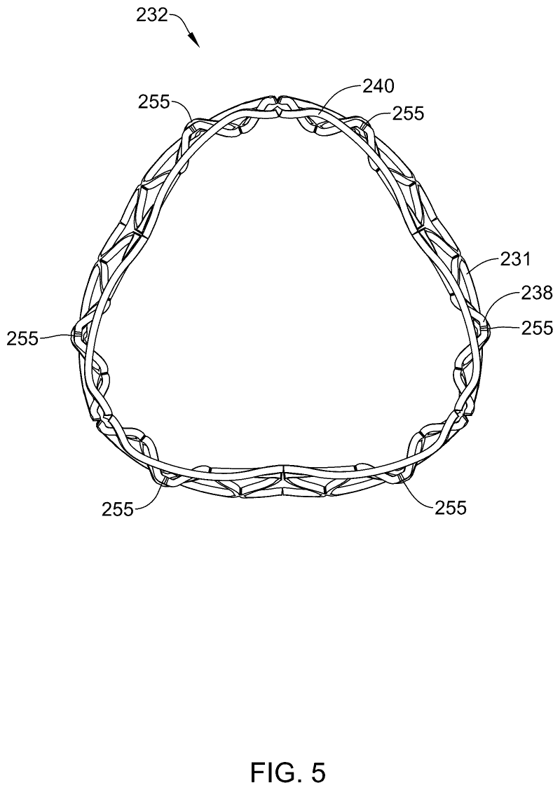

FIG. 5 is a top down view of the expandable framework 232 of FIG. 3, without the valve leaflets or inner skirt. The expandable framework 232 is in the expanded configuration looking upstream from the stabilization arches 240 downwards through the expandable framework 232. In some embodiments, the expandable framework 232 may have a substantially circular cross-section. In some embodiments, the expandable framework 232 can have a non-circular (e.g., D-shaped, triangular, elliptical, etc.) cross-section. The radially outwardly extending upper crowns 238 and the free points 255 can be seen extending radially outward from the remainder of the struts 231. In the embodiment shown, six free points 255 are spaced apart circumferentially around the expandable framework 232.

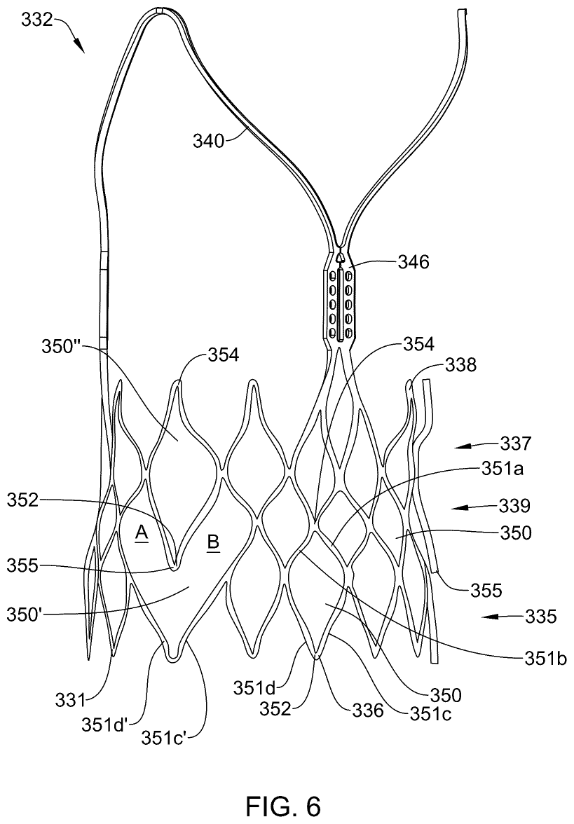

The embodiment shown in FIG. 6 illustrates a second expandable framework 332 defining a second configuration of free points 355. While FIG. 6 is only a partial cross-sectional view, it will be understood that the expandable framework 332 defines a circumferential structure similar to that in FIG. 3, but with a different cell structure. This expandable framework 332 may also be used in a replacement heart valve implant with leaflets and an inner and/or outer skirt. FIG. 6 shows the expandable framework 332 as further including a third circumferential row 339 of cells 350 positioned axially between and circumferentially offset from first and second circumferential rows 335, 337 of cells. The cells in the first, second and third circumferential rows are diamond shaped with a majority of the cells 350 in the expandable framework 332 having an upper right strut 351a and an upper left strut 351b meeting at the upper point 354 of the cell 350, and a lower right strut 351c and lower left strut 351d meeting at a lower point 352 of the cell. In some embodiments, 80% to 95% of the cells 350 in the expandable framework 332 have the upper right and upper left struts 351a, 351b as described above. The upper right and left struts of each cell in the first circumferential row 335 are the same as the lower left and right struts, respectively, of adjacent cells in the third circumferential row 339. At least a first cell 350′ in the first circumferential row 335 is devoid of upper left and right struts such that the first cell 350′ defines a V-shaped cell combined with first (A) and second (B) adjacent cells in the third circumferential row 339. The lower point 352 of a first cell 350'′ in the second circumferential row 337 that is axially aligned with the first cell 350′ in the first circumferential row 335, is a free point 355 that extends radially outward of any struts 331 below it in the upstream direction.

Some of the lower points 352 of the cells 350 in the first circumferential row 335 define the lower crowns 336 and some of the upper points 354 of the cells in the second circumferential row 337 define the upper crowns 338. The upper points 354 defining upper crowns 338 may be bent radially outward. The expandable framework 332 may include at least three free points 355. In the embodiment shown in FIG. 6, three free points 355 are spaced apart circumferentially around the expandable framework 332, with one free point 355 positioned under each stabilization arch 340. In other embodiments, four or more free points 355 may be positioned circumferentially around the expandable framework 332. In some embodiments, a free point 355 may be positioned under a commissure 346.

The free points 355 are configured to trap or pin a leaflet of the native heart valve against the lower struts of the expandable framework. The free points 355 may apply a greater force to the anatomy, and this increased force may increase the total radial force applied by the expandable framework 332 on the anatomy to reduce or prevent movement of the expandable framework and the replacement valve including the framework.

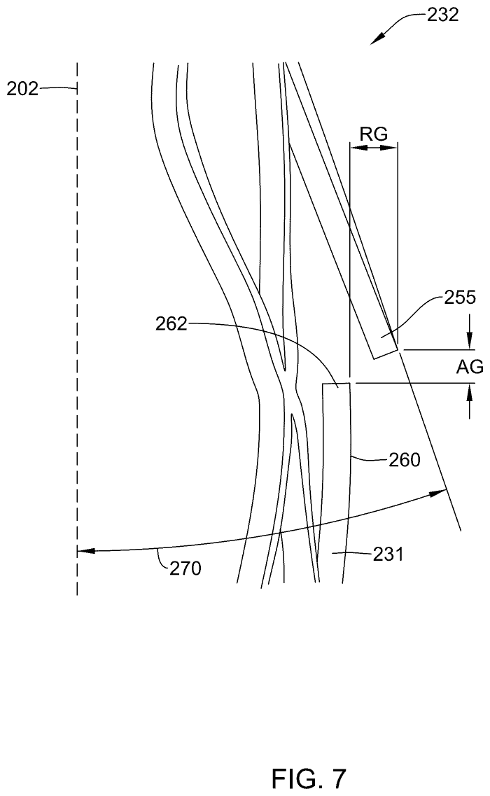

FIG. 7 is a close-up side view of the free point 255 of the expandable framework 232 of FIG. 3. The outer surface of the free point 255 may extend radially outward beyond the radially outer surface of any strut below the free point 255 in the axial direction. This extension may define a radial gap RG of between 2 millimeters (mm) (0.079 inches) and 6 mm (0.236 inches) from the radial outer surface of the tip of the free point 255 to the radially outer surface 260 of the strut 231 below the free point 255. The extension of the free point 255 may define an angle, indicated by arrow 270, of 20 degrees to 60 degrees relative to the central longitudinal axis 202. This extension of the free point 255 may also create an axial gap AG of between 0.7 mm (0.028 inches) and 2 mm (0.079 inches) between the upper surface 262 of the strut 231 below the free point 255 and the outermost point of the free point 255. The combination of the radial gap RG and the axial gap AG creates space configured to receive the free edge of one of the native heart valve leaflets and trap or pin that valve leaflet against the expandable framework when expanded within the native heart valve.

The free point 355 in the expandable framework 332 shown in FIG. 6 may extend radially outward from the outer surface of the remainder of the struts at similar angles and measurements as described above. For example, the free point 355 may extend radially outward from the outer surface of each of the lower right strut 351c′ and the lower left strut 351d′ of the cell 350′ below the free point at a distance of 2.0 mm (0.079 inches) to 6.0 mm (0.236 inches), forming the radial gap RG. The free point 355 may also extend at an angle of 20 degrees to 60 degrees from the central longitudinal axis of the expandable framework 332.

In some embodiments, the expandable framework 232, 332 may have an outer extent of about 23 mm (0.906 inches), about 25 mm (0.984 inches), about 27 mm (1.063 inches), about 30 mm (1.181 inches), etc. in an unconstrained configuration (e.g., in the expanded configuration). In some embodiments, the expandable framework 232, 332 may have an outer extent of about 10 mm (0.394 inches), about 9 mm (0.354 inches) about 8 mm (0.315 inches), about 7 mm (0.276 inches), about 6 mm (0.236 inches), etc. in the collapsed configuration. Other configurations are also contemplated.

During delivery, the replacement heart valve implant may be disposed within a delivery catheter system with the expandable framework 232, 332 in a collapsed configuration. In use, the delivery catheter system may be advanced percutaneously through the vasculature to a position adjacent to a treatment site. For example, the delivery catheter system may be advanced through the vasculature and across the aortic arch upstream to a position adjacent to a defective native heart valve such as the aortic valve. Alternative approaches to treat a defective aortic valve and/or other heart valve(s) are also contemplated with the medical device system. After navigating the delivery catheter system to the treatment site, the replacement heart valve implant may be released. When unconstrained by the delivery catheter system, the expandable framework 232, 332 may be configured to shift from the collapsed configuration to an expanded configuration. The replacement heart valve implant may be deployed within the native heart valve (e.g., the native heart valve is left in place and not excised).

The replacement heart valve may include an expandable framework 232, 332 and replacement valve leaflets 234 secured thereto, as shown in FIG. 3. The expandable framework 232, 332 may be as described above, including a plurality of struts 231, 331 defining a lattice structure around a central longitudinal axis, the lattice structure defining an inflow end 201 and an opposite outflow end 203, the plurality of struts 231, 331 defining a plurality of stabilization arches 240, 340 extending downstream from the outflow end 203 of the lattice structure. The plurality of struts 231, 331 defines a plurality of lower crowns 236, 336 defining the inflow end 201 and a plurality of upper crowns 238, 338 defining the outflow end 203. The lattice structure includes a first circumferential row 235, 335 of cells defining the lower crowns 236, 336 and inflow end 201 and a second circumferential row 237, 337 of cells 250, 350 defining the upper crowns 238, 338 and the outflow end 203, where the cells of the first and second circumferential rows each have a lower point 252, 352 and an upper point 254, 354. The lower point of at least some of the cells in the second circumferential row defines a free point 255, 355 devoid of attachment to any other strut and extends radially outward beyond a radially outer surface of all struts immediately below in an axial direction.

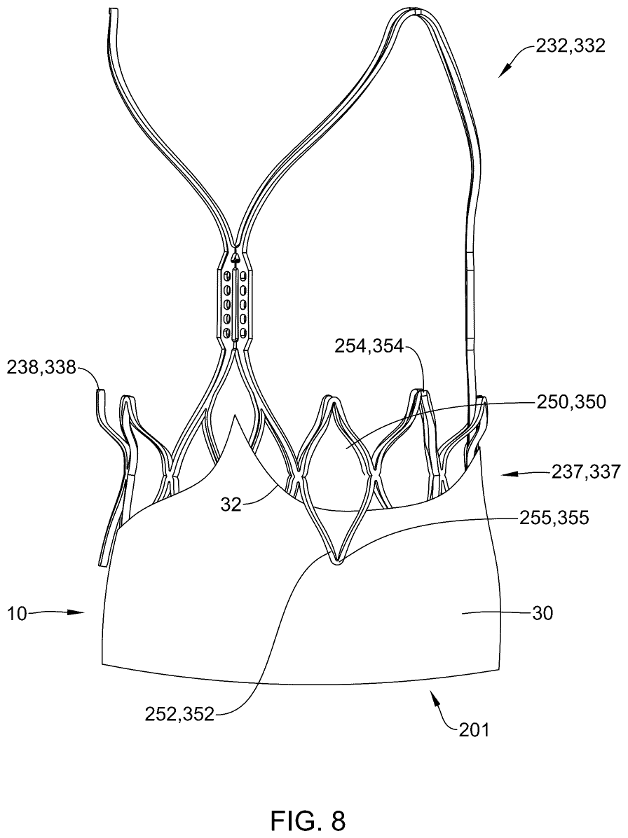

The method further includes positioning the expandable framework over the free edge 32 of the native valve leaflet 30 to pin the free edge 32 of at least one leaflet of the native heart valve under the free point 255, 355, between the free point and the struts below the free point, as shown in FIG. 8. The replacement heart valve is advanced in a compressed configuration and is expanded when in place within the native heart valve.

The materials that can be used for the various components of the medical device system and the various elements thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the expandable framework, the inner skirt, the outer skirt, the plurality of leaflets, and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material. In one embodiment, the expandable framework 232, 332 may be formed from laser cut nitinol heat set with the free points 255, 355 in the radially extended configuration.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyisobutylene (PIB), polyisobutylene polyurethane (PIBU), polyurethane silicone copolymers (for example, Elast-Eon® from AorTech Biomaterials or ChronoSil® from AdvanSource Biomaterials), ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof, may be made of a material that does not substantially distort the image and create substantial artifacts (i.e., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure, such as for an inner and/or outer skirt. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An expandable framework for use in a replacement heart valve implant, comprising:
a plurality of struts defining a lattice structure around a central longitudinal axis, the lattice structure defining an inflow end and an opposite outflow end, the plurality of struts defining a plurality of stabilization arches extending downstream from the outflow end of the lattice structure;
wherein the plurality of struts defines a plurality of lower crowns defining the inflow end and a plurality of upper crowns defining the outflow end;
wherein the lattice structure includes a first circumferential row of cells defining the lower crowns and the inflow end and a second circumferential row of cells defining the upper crowns and the outflow end, wherein the cells of the first and second circumferential rows each have a lower point and an upper point; and
wherein the lower point of at least some of the cells in the second circumferential row is a free point devoid of attachment to any other strut and extends radially outward beyond a radially outer surface of any strut immediately below in an axial direction.

2. The expandable framework of claim 1, wherein some of the lower points of the cells in the first circumferential row define the lower crowns and some of the upper points of the cells in the second circumferential row define the upper crowns, wherein the upper points defining upper crowns are bent radially outward.

3. The expandable framework of claim 1, wherein three or more free points are spaced apart around the lattice structure.

4. The expandable framework of claim 1, wherein an outer surface of each free point extends radially outward 2.0 mm (0.079 inches) to 6.0 mm (0.0236 inches) beyond the radially outer surface of any strut below in the axial direction.

5. The expandable framework of claim 4, wherein an axial gap of 0.7 mm (0.028 inches) to 2 mm (0.079 inches) is defined between each free point and the strut immediately below in the axial direction.

6. The expandable framework of claim 1, wherein each free point extends at an angle of 20 degrees to 60 degrees from the central longitudinal axis of the expandable framework.

7. The expandable framework of claim 1, wherein the cells in the first and second circumferential rows are diamond shaped with upper left and right struts meeting at the upper point of each cell and lower left and right struts meeting at the lower point of each cell, wherein some cells in the first circumferential row have a right side connection point joining the upper and lower right struts and a left side connection point joining the upper and lower left struts, where the right side connection point is also joined with the left side connection point of a circumferentially adjacent cell to form an X shaped join, wherein some circumferentially adjacent cells in the first circumferential row are devoid of the X shaped join wherein the upper right strut of a first cell is joined only with the upper left strut of an adjacent second cell, and the lower right strut of the first cell is joined only with the lower left strut of the second cell such that the joined upper right and left struts of the first and second cells defines the free point of one of the cells in the second circumferential row.

8. The expandable framework of claim 7, wherein some of the lower points of the cells in the first circumferential row define the lower crowns and wherein some of the upper points of the cells in the second circumferential row define the upper crowns, wherein the upper points defining upper crowns are bent radially outward.

9. The expandable framework of claim 7, wherein three or more free points are spaced apart around the lattice structure.

10. The expandable framework of claim 7, wherein an outer surface of each free point extends radially outward 2.0 mm (0.079 inches) to 6.0 mm (0.236 inches) beyond the radially outer surface of any strut immediately below in the axial direction.

11. The expandable framework of claim 10, wherein an axial gap of 0.7 mm (0.028 inches) to 2 mm (0.079 inches) is defined between each free point and the strut immediately below in the axial direction.

12. The expandable framework of claim 7, wherein each free point extends at an angle of 20 degrees to 60 degrees from the central longitudinal axis of the expandable framework.

13. The expandable framework of claim 1, further comprising a third circumferential row of cells between and circumferentially offset from the first and second circumferential rows of cells, wherein the cells in the first, second and third circumferential rows are diamond shaped with a majority of cells having upper left and right struts meeting at the upper point of the cell and left and lower right struts meeting at the lower point of the cell, wherein the upper left and right struts of each cell in the first circumferential row are the lower right and left struts, respectively, of adjacent cells in the third circumferential row, wherein at least a first cell in the first circumferential row is devoid of upper left and right struts such that the first cell defines a V-shaped cell with first and second adjacent cells in the third circumferential row, and the free point is defined by the lower point of a first cell in the second circumferential row axially aligned with the first cell in the first circumferential row.

14. The expandable framework of claim 13, wherein at least three free points are spaced apart around the lattice structure.

15. The expandable framework of claim 13, wherein an outer surface of each free point extends radially outward 2.0 mm (0.079 inches) to 6.0 mm (0.236 inches) beyond the radially outer surface of any strut immediately below in the axial direction, and each free point extends at an angle of 20 degrees to 60 degrees from the central longitudinal axis of the expandable framework.

16. A replacement heart valve comprising:
the expandable framework according to claim 1; and
a plurality of valve leaflets disposed within a central lumen of the lattice structure and coupled to the expandable framework.

17. A replacement heart valve comprising:
an expandable framework including a plurality of struts defining at least first and second circumferential rows of diamond shaped cells around a central longitudinal axis defining an inflow end and an opposite outflow end, the plurality of struts defining a plurality of stabilization arches extending downstream from the outflow end; and
a plurality of valve leaflets disposed within a central lumen of the expandable framework and coupled to the expandable framework;
wherein the first circumferential row of cells defines a plurality of lower crowns at the inflow end, and the second circumferential row of cells defines a plurality of upper crowns at the outflow end, wherein the cells of the first and second circumferential rows each have a lower point and an upper point;
wherein the lower point of at least some of the cells in the second circumferential row is a free point devoid of attachment to any other strut and extends radially outward beyond a radially outer surface of any strut immediately below in an axial direction.

18. The replacement heart valve of claim 17, wherein the diamond shaped cells in the first and second circumferential rows have upper left and right struts meeting at the upper point of the cell and lower left and right struts meeting at the lower point of the cell, wherein some cells in the first circumferential row have a right side connection point joining the upper and lower right struts and a left side connection point joining the upper and lower left struts, where the right side connection point is also joined with the left side connection point of a circumferentially adjacent cell to form an X shaped join, wherein some circumferentially adjacent cells in the first circumferential row are devoid of the X shaped join wherein the upper right strut of a first cell is joined only with the upper left strut of an adjacent second cell, and the lower right strut of the first cell is joined only with the lower left strut of the second cell such that the joined upper right and left struts of the first and second cells defines the free point of one of the cells in the second circumferential row.

19. The replacement heart valve of claim 17, further comprising a third circumferential row of cells between and circumferentially offset from the first and second circumferential rows of cells, wherein the cells in the first, second and third circumferential rows are diamond shaped with a majority of cells having upper left and right struts meeting at the upper point of the cell and lower left and right struts meeting at the lower point of the cell, wherein the upper left and right struts of each cell in the first circumferential row are the lower right and left struts, respectively, of adjacent cells in the third circumferential row, wherein at least a first cell in the first circumferential row is devoid of upper left and right struts such that the first cell defines a V-shaped cell with first and second adjacent cells in the third circumferential row, and the free point is defined by the lower point of a first cell in the second circumferential row axially aligned with the first cell in the first circumferential row.

20. A method of implanting a replacement heart valve within a patient's native heart valve, comprising:
advancing a replacement heart valve through a patient's vasculature and upstream into the patient's native heart valve, the replacement heart valve including:
an expandable framework and replacement valve secured thereto, the expandable framework including a plurality of struts defining a lattice structure around a central longitudinal axis, the lattice structure defining an inflow end and an opposite outflow end, the plurality of struts defining a plurality of stabilization arches extending downstream from the outflow end of the lattice structure;
wherein the plurality of struts defines a plurality of lower crowns defining the inflow end and a plurality of upper crowns defining the outflow end;
wherein the lattice structure includes a first circumferential row of cells defining the lower crowns and the inflow end and a second circumferential row of cells defining the upper crowns and the outflow end, wherein the cells of the first and second circumferential rows each have a lower point and an upper point;
wherein the lower point of at least some of the cells in the second circumferential row is a free point devoid of attachment to any other strut and extends radially outward beyond a radially outer surface of all struts immediately below in an axial direction; and
positioning the expandable framework to pin at least one leaflet of the native heart valve between the free point and the struts below the free point.
